(19) United States
(12) Patent Application Publication
(10) Pub. No.: US 2024/0148270 A1
(43) Pub. Date: May 9, 2024

(54) METHOD FOR EXTRACTING RESPIRATORY INFORMATION FROM A BIO-IMPEDANCE SIGNAL

(71) Applicant: Onera Technologies B.V., Eindhoven (NL)

(72) Inventors: Alessandro Rossi, Eindhoven (NL); Hartmut Schneider, Eindhoven (NL)

(21) Appl. No.: 18/281,489

(22) PCT Filed: Mar. 9, 2022
(86) PCT No.: PCT/EP2022/055964

§ 371 Date: Sep. 11, 2023

(30) Foreign Application Priority Data
Mar. 12, 2021 (EP) 21162254.3

(51) Int. Cl. A61B 5/08; A61B 5/00; A61B 5/087

(52) U.S. Cl. CPC A61B 5/0809; A61B 5/0816; A61B 5/087; A61B 5/725; A61B 5/7278; A61B 5/053

(57) ABSTRACT

This application relates to a method (S0, S0′) for extracting respiratory information from a bio-impedance signal. A first method (S0) relates to determining a respiratory effort signal from a bio-impedance signal by means of Savitzky-Golay low-pass filtering. A second method (S0′) relates to determining a respiratory flow signal from a noise filtered respiratory effort signal not limited to being extracted from a bio-impedance signal, which second method (S0′) implements Savitzky-Golay differentiation. This application also relates to a computer program comprising instructions which, when executed by a computing device, cause the computing device to carry out the first or second method (S0, S0′).

### TECHNICAL FIELD

The present invention relates to a method for extracting respiratory information from a bio-impedance signal, and in particular a method for extracting respiratory information from a bio-impedance signal by means of computationally efficient filters.

### BACKGROUND

When assessing the health condition of a subject, it is common to monitor the subject's respiration. One way of doing so relies on extracting respiratory information from a subject's bio-impedance signals, in particular when acquired using electrodes placed on the torso of a subject. This is possible since the bio-impedance signal is dependent on the content and distribution of tissues and materials along the path of an injected current; hence, air flowing in and out of the lungs and bronchus will cause measurable variations in accordance with the respiratory behavior of the subject.

However, the bio-impedance signal also includes disturbances that are mostly related to other tissue components (skin, muscle, fat, blood) and their periodic motion throughout respiratory and cardiac cycles. Therefore, it is challenging to separate respiratory information from disturbances present in the bio-impedance signal, which can often be in partially overlapping frequency bands.

Thus, there is a need for a method for extracting respiratory information from a bio-impedance signal in an accurate and computationally efficient manner.

### SUMMARY

It is an object of the present invention to provide a method for extracting accurate and precise respiratory information from a bio-impedance signal in a computationally efficient manner.

The invention is primarily based on the inventor's realization that using a Savitzky-Golay low pass filter incorporating a 3^{rd} degree polynomial is particularly useful for enabling accurate and precise respiratory information to be extracted from bio-impedance signals. The reason for this is that such a Savitzky-Golay filter is able to smooth data sets for increasing precision of the data without distorting it to unacceptable levels. It is therefore useful in applications of extracting qualitative information from relatively noisy signals, such as extracting respiratory information from subject measurement signals, e.g. bio-impedance signals. Further, Savitzky-Golay filters incorporating a 3^{rd} degree polynomial have relatively little computational cost as compared to other smoothing techniques. It is therefore useful in applications mandating rapid computation, such as real-time monitoring of respiratory information of a subject.

Moreover, when differentiating a respiratory effort signal characterized by little noise by means of a Savitzky-Golay derivative kernel incorporating a 2^{nd} degree polynomial fit, it has been identified that additional accurate and precise respiratory information representing a respiratory flow signal can be extracted. Obtaining a respiratory effort signal characterized by little noise is obtained as a consequence of applying a Savitzky-Golay low pass filter incorporating a 3^{rd} degree polynomial fit on a bio-impedance signal. This filtering cascade allows our method to track relative breath-to-breath amplitude changes in both respiratory effort and respiratory flow and comparing such effort and flow relative changes to each other in order to assess whether upper airway obstructions are present. Moreover, it would also be possible to use a respiratory effort signal not necessarily obtained by the Savitzky-Golay low-pass filter applied on a bio-impedance signal; it could be sufficient to use a respiratory effort signal and noise filtering it. In view of this, a method according to the present invention may be provided in two alternatives, the first and second aspect of the invention which are described in the following.

According to a first aspect of the invention, a method for extracting respiratory information from a bio-impedance signal is provided. The method comprises the steps of: providing a bio-impedance signal of a subject; filtering the bio-impedance signal using a Savitzky-Golay low-pass filter to provide first respiratory information representing a respiratory effort signal, wherein the Savitzky-Golay low-pass filter incorporates a 3^{rd} degree polynomial fit.

By this, accurate and precise first respiratory information representing a respiratory effort signal may be extracted from a bio-impedance signal in a computationally efficient manner. In particular, the method efficiently retrieves respiratory information using significantly less processing steps than previously proposed methods, while eliminating cardiac oscillations from the bio-impedance signal, and thereby resulting in properly de-noised respiratory information.

The bio-impedance signal may be measured by means of electrodes arranged on the torso of a subject. The electrodes may be two, three, four, five, six or more in total and distributed over the torso so as to enable a bio-impedance signal to be captured according to different measurement configurations. According to one preferred embodiment, the bio-impedance signal is captured by electrodes arranged on a subject's torso in a tetrapolar configuration.

According to one further embodiment, the method comprises the step of differentiating the respiratory effort signal with a Savitzky-Golay first derivative kernel to provide second respiratory information representing a respiratory flow signal, wherein the Savitzky-Golay first derivative kernel incorporates a 2^{nd} degree polynomial fit. By this, additional respiratory information may be retrieved. Furthermore, this way of extracting respiratory information representing a respiratory flow signal is both computationally efficient and provides accurate and precise information.

According to a second aspect of the invention, a method for extracting respiratory information from respiratory measurements of a subject, preferably from a bio-impedance signal. The method comprises the steps of: providing a noise filtered respiratory effort signal, preferably a respiratory effort signal extracted from a bio-impedance signal; differentiating the noise filtered respiratory effort signal with a Savitzky-Golay first derivative kernel to provide second respiratory information representing a respiratory flow signal, wherein the Savtizky-Golay first derivative kernel incorporates a 2^{nd} degree polynomial fit.

By this, accurate and precise respiratory information representing a respiratory flow signal may be extracted from a bio-impedance signal in a computationally efficient manner. In particular, the method efficiently retrieves respiratory information using significantly less processing steps than previously proposed methods, while eliminating cardiac oscillations from the bio-impedance signal, and thereby resulting in properly de-noised respiratory information.

By respiratory measurements, it is meant measurements that result in some effort signal. Examples of such respiratory measurements include: respiratory thoracic and/or abdominal belts (which can in turn be based for instance on inductive plethysmography, on resistive strain gauges), esophageal pressure transducers, accelerometer-based motion signals estimated on the torso, and more. The respiratory measurements of a subject may preferably be bio-impedance measurements resulting in bio-impedance signal. The bio-impedance signal may be measured by means of electrodes arranged on the torso of a subject. The electrodes may be two, three, four, five, six or more in total and distributed over the torso so as to enable a bio-impedance signal to be captured according to different measurement configurations. According to one preferred embodiment, the bio-impedance signal is captured by electrodes arranged on a subject's torso in a tetrapolar configuration.

According to one further embodiment of the second aspect of the invention, the respiratory effort signal is extracted from the respiratory measurement by filtering the bio-impedance signal using a Savitzky-Golay low-pass filter incorporating a 3^{rd} degree polynomial fit. This is one way of obtaining a noise filtered respiratory effort signal. This in particular may allow for more accurate and precise first respiratory information to be extracted in a computationally efficient manner.

In the following, embodiments applicable to both the first and second aspect of the invention are summarized.

According to one embodiment, the method may comprise a step of filtering the respiratory effort signal using a high-pass filter to obtain a baseline filtered respiratory effort signal. This allows the low frequency baseline of the bio-impedance signal to be removed, while keeping in the signal transient changes in the baseline itself, thanks to the fact that the cutoff frequency of the high-pass filter is selected so as to preserve predetermined frequency components expected to be relevant in respiratory assessment, which for instance can be related to residual changes in lung volume, dynamic hyperinflation leading to arousals during sleep, repetitive Vansalva maneuvers, and other similar events related to breathing abnormalities (eg: COPD, obstructive sleep apnea, and others).

According to one embodiment, the Savtisky-Golay filter or filters are adaptively updated based on an instantaneous respiratory frequency. This has the advantage of allowing accurate and precise respiratory information to be extracted from a bio-impedance signal even if the subject's respiration frequency changes overtime, either smoothly or abruptly. It may also facilitate accurate and precise respiratory information to be extracted from a bio-impedance signal which may be related to the subject's respiration frequency. The instantaneous respiratory frequency may be measured by a respiratory monitoring device.

According to one embodiment, the instantaneous respiratory frequency is estimated from the bio-impedance signal. This provides the advantage that no external respiratory monitoring device is required. The instantaneous respiratory frequency may be estimated from the bio-impedance signal by means of peak and/or valley detection.

The method may incorporate the steps of detecting a breath-by-breath breathing rate, BR, from the bio-impedance, BI, signal; performing a moving average of the resulting BR-signal with non-overlapping time windows of frame lengths in the interval of 15-30 seconds, which resulting BR-signal is denoted as ABR-signal, and applying a multiplier M so that at any point in time along the BI-signal, the adaptive frame length of the Savitzky-Golay smoothing filter is equal to M*(1/ABR), thus resulting in an adaptively smoothed bio-impedance, ASBI, signal, wherein said M is a value selected in the interval of 0.10-0.30.

Further, the high-pass filter may be any known high-pass filter, for example Chebyshev filter, Butterworth filter, or Elliptic filter. Other known high-pass filters may be used also. In a specific example, a zero-phase Butterworth filter may be used. By this, it is possible to obtain a relatively steep attenuation of frequency components below the cut-off frequency while not distorting the remainder of the signal in any significant manner. More specifically, the high-pass zero-phase Butterworth filter may be a 2^{nd} order high-pass Butterworth filter. By this, it is possible to achieve sufficient quality in the baseline-filtered bio-impedance signal while ensuring sufficiently low computational steps, which is beneficial in terms of computational speed.

According to one embodiment, the high-pass filter has a cutoff frequency between 0.001-0.1 Hz, preferably between 0.01-0.05 Hz. By this, the baseline of the bio-impedance signal is sufficiently filtered. The cutoff frequency may be in the interval of 0.001-0.01 Hz, 0.01-0.02 Hz, 0.02-0.03 Hz, 0.03-0.04 Hz, 0.04-0.05 Hz, 0.05-0.06 Hz, 0.06-0.07 Hz, 0.07-0.08 Hz, 0.08-0.09 Hz, 0.09-0.1 Hz.

According to one embodiment, the Savitzky-Golay low-pass filter is applied on subsets of data points of the bio-impedance signal which are filtered using a frame length between 1-2 seconds, preferably between 1.3-1.7 seconds. By having such a frame length, the frame length is adequate in order to keep most of the respiratory (spectral) information in the estimated effort signal while also providing adequate noise rejection (for both cardiac oscillations and high frequency noise) and smoothing. The frame length may be 1.1-1.2 seconds, 1.2-1.3 seconds, 1.3-1.4 seconds, 1.4-1.5 seconds, 1.5-1.6 seconds, 1.6-1.7 seconds, 1.7-1.8 seconds, 1.8-1.9 seconds, or 1.9-2.0 seconds.

According to one embodiment, the Savitzky-Golay first derivative kernel is applied on subsets of datapoints of the respiratory effort signal which are filtered using a frame length between 0.5-1.5 seconds, preferably between 0.8-1.2 seconds. By having such a frame length, the frame length is adequate in order to keep most of the respiratory (spectral) information in the estimated effort signal while also providing adequate noise rejection (for both cardiac oscillations and high frequency noise) and smoothing. The frame length may be 0.5-0.6 seconds, 0.6-0.7 seconds, 0.7-0.8 seconds, 0.8-0.9 seconds, 0.9-1.0 seconds, 1.0-1.1 seconds, 1.1-1.2 seconds, 1.2-1.3 seconds, 1.3-1.4 seconds, or 1.4-1.5 seconds.

According to one embodiment, the Savitzky-Golay derivative kernel is applied on the respiratory effort signal without first having been normalized.

According to one embodiment, the respiratory effort signal and/or the respiratory flow signal are normalized. Both signals may be normalized.

According to a third aspect of the invention, a computer program is provided. The computer program comprises instructions which, when executed by a computing device, cause the computing device to carry out the method according to the first or second aspect or any embodiments thereof.

The computing device may be a portable computing device such as a smartphone, a smartwatch, a tablet, or a laptop. The computing device may alternatively be a workstation or a server. In case of a server, the program code may be controlled from an interface running on a remote computing device. The program code may be executed by means of cloud computing.

According to a fourth aspect of the invention, a program readable storage medium is provided. The storage medium stores the computer program according to the third aspect of the invention.

According to a fifth aspect of the invention, a device for extracting respiratory information from a bio-impedance signal is provided. The device comprises a processor configured to execute the method according to the first aspect of the invention, or any embodiments thereof. The device may also comprise a housing for housing the processor. The device may comprise other electronics components such as one or more program readable storage mediums, electrodes and a circuit board configured to work together with the processor so as to enable to device to carry out the method according to the first aspect of the invention or any embodiments thereof. The device may also comprise a power source, such as a battery, or means to connect to an exterior power source or a power outlet.

According to one embodiment, the device comprises a program readable storage medium configured to store extracted respiratory information and/or Savitzky-Golay filter parameters, and/or in the case the Savitzky-Golay filter or filters are configured to be adaptively updated, updated Savitzky-Golay filter parameters. By this, the respiratory information may then be accessed at a later time. Further, the memory profile may be configured to store profiles for different sets of Savitzky-Golay filter parameters for different respiratory conditions.

According to one embodiment, the device is a wearable patch unit. By this, the device may be configured to execute the method locally.

According to one embodiment, the device comprises a communications module configured to enable the device to receive and process filter control data, which control data specify control data of the Savitzky-Golay filter or filters. By this, further control of the Savitzky-Golay filter or filters may be enabled.

The method and/or device above may be used for assisting in respiratory assessments, including but not limited to sleep scoring, sleep apnea detection, lung disease monitoring, telemedicine, triaging. By assisting in respiratory assessments, it may mean that the method and/or device only provide respiratory information which a medical professional may use as aid when making a diagnosis. The method and/or device may then continue to provide respiratory information to enable continued respiratory assessments to be made in accordance with the established diagnosis. The method and/or device may exclude any step of evaluating what the extracted respiratory assessments in terms of a diagnosis; and leave this step for a professional to evaluate.

In addition, the bioimpedance signal may be smoothed in an adaptive manner. This may incorporate a step of detecting a breath-by-breath breathing rate (BR) from the bioimpedance signal and performing a moving average of the resulting BR-signal with non-overlapping time windows of frame lengths in the interval of 15-20 seconds, 20-25 seconds, 25-30 seconds, or a combination of said intervals. The time windows may span a plurality of breaths. The resulting instantaneously averaged BR signal may be denoted as ABR signal. Further, this may incorporate a step of applying a multiplier M so that at any point in time along the bioimpedance signal, the adaptive frame length of the Savitzky-Golay smoothing filter (3^{rd} order) is equal to M*(1/ABR). M may be a value selected in the interval of 0.10-0.15, 0.15, 0.20, 0.20-0.25, or 0.25-0.30.

The invention is defined by the appended independent claims, with embodiments being set forth in the appended dependent claims, in the following description and in the drawings.

### BRIEF DESCRIPTION OF THE DRAWINGS

The invention will in the following be described in more detail with reference to the enclosed drawings, wherein:

FIG. 1 shows a chart of the method according to the first and second aspect of the invention respectively—dashed segments indicate a further embodiment(s);

FIG. 2 shows diagrams of a provided bio-impedance signal and the resulting respiratory effort signal and respiratory flow signal obtained by the present invention according to one embodiment of the invention;

FIG. 3 shows a block diagram of the components of a device according to one embodiment of the invention wherein dashed segments indicate a further embodiment of the invention;

FIG. 4 shows a chart of the method according to one embodiment of the invention.

### DESCRIPTION OF EMBODIMENTS

The present invention will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. In the drawings, like numbers refer to like elements.

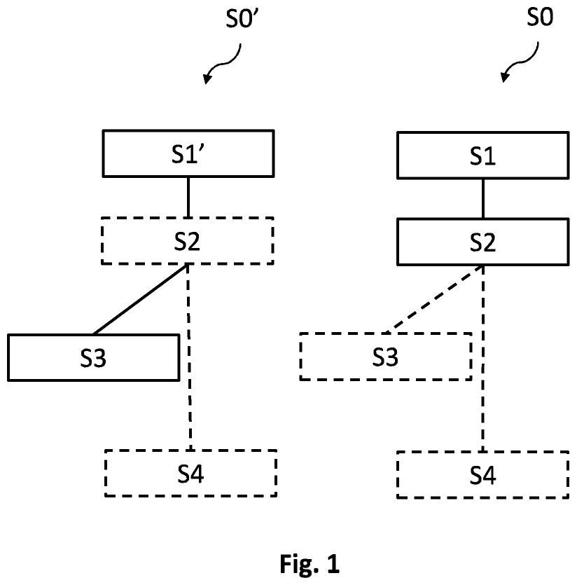

FIG. 1 shows a chart of the method according to the first and second aspect of the invention respectively—dashed segments indicate a further embodiment(s). The methods differ in that one method S0 relates to providing a respiratory effort signal while the other method S0′ relates to providing a respiratory flow signal.

The method S0 according to one embodiment comprises the step of providing S1 a bio-impedance signal of a subject. The bio-impedance signal is measured by means of electrodes arranged on the torso of the subject, preferably in a tetrapolar configuration. The method further comprises a step of filtering S2 the bio-impedance signal using a Savitzky-Golay low-pass filter to provide first respiratory information representing a respiratory effort signal. The Savitzky-Golay low-pass filter incorporates a 3^{rd} order polynomial fit. Further, the Savtizky-Golay low-pass filter is using a frame length between 1-2 seconds.

By this, it is possible to extract respiratory information which is both accurate and precise. Further, it is a very computationally efficient method to extract such respiratory information and may advantageously be used in applications mandating rapid computation, such as real-time monitoring of respiratory information.

According to one further embodiment, which is indicated in FIG. 1, the method S0 comprises a step of differentiating S3 the respiratory effort signal with a Savitzky-Golay first derivative kernel to provide second respiratory information representing a respiratory flow signal. The Savtizky-Golay first derivative kernel incorporates a 2^{nd} order polynomial fit. The Savitzky-Golay first derivative kernel is applied on subsets of datapoints of the respiratory effort signal which are determined using a frame length between 0.5-1.5 seconds. Further, the Savtizky-Golay first derivative kernel is applied on the respiratory effort signal, optionally without it first having been normalized. The resulting respiratory flow signal may then be normalized.

The method S0′ according to one embodiment comprises the step of providing S1′ a noise filtered respiratory effort signal. Preferably, the noise filtered respiratory effort signal is a respiratory effort signal extracted from a bio-impedance signal, which respiratory effort signal then is noise filtered if so required. The method further comprises a step of differentiating S3 the noise filtered respiratory effort signal with a Savitzky-Golay first derivative kernel to provide second respiratory information representing a respiratory flow signa. The Savtizky-Golay first derivative kernel incorporates a 2^{nd} degree polynomial fit.

According to one further embodiment, the respiratory effort signal is extracted from the bio-impedance signal by filtering S2 the bio-impedance signal using a Savitzky-Golay low-pass filter incorporating a 3^{rd} degree polynomial fit.

Either of the methods S0, S0′ comprises in further embodiments a step S4 of filtering the respiratory effort signal using a high-pass filter to obtain a baseline-filtered bio-impedance signal. The high-pass filter used is a high-pass zero-phase Butterworth filter of 5^{th} order with a low cut-off frequency in the range of 0.001-0.1 Hz. Other high-pass filter may be viable depending on parameters.

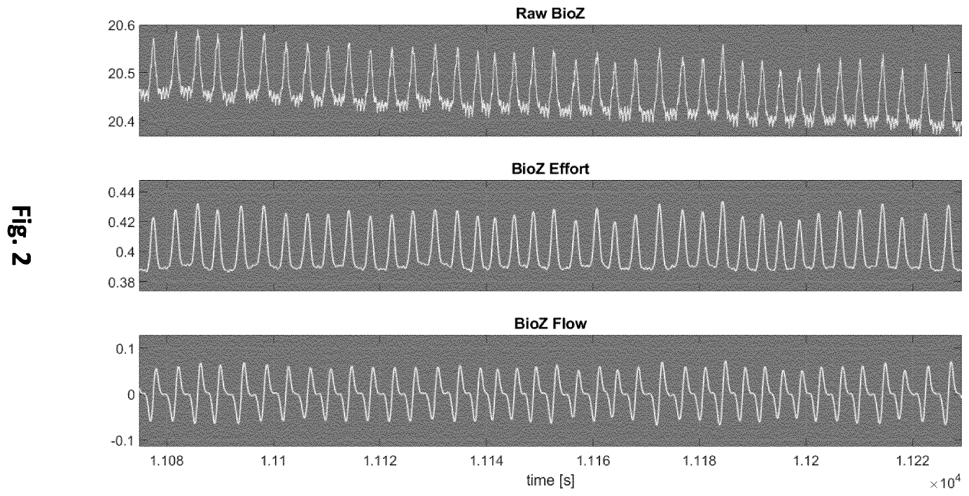

FIG. 2 shows diagrams of a provided bio-impedance signal and the resulting respiratory effort signal and respiratory flow signal obtained by the present invention according to one embodiment of the invention. The top graph shows a provided bio-impedance signal. The middle graph shows the resulting respiratory effort signal after being normalized in the [0, 1] range. This particular respiratory effort shown is a segment from a signal which was achieved with a low-pass Savtizky-Golay filter incorporating a 3^{rd} order polynomial fit and a frame-length of 1.55 seconds, and a high-pass zero-phase Butterworth filter of the 2^{nd} degree with a cut-off frequency of 0.02 Hz and. The bottom graph shows the resulting respiratory flow signal after being normalized in the [−1, 1] range. This particular flow signal was achieved by filtering the respiratory effort signal in the middle graph (shown in arbitrary units, but still having a non-zero baseline) using a Savitzky-Golay kernel incorporating a 2^{nd} degree polynomial fit with a frame length of 1 second.

The choice of a 2^{nd} degree 1^{st}-derivative Savitzky-Golay filter cascaded to the aforementioned 3^{rd} degree low-pass Savitzky-Golay filter enables the derivation of a smooth respiratory flow signal from the bio-impedance signal, which still retains fast variations possibly associated to clinically relevant breathing events.

According to one aspect of the invention, a computer program is provided also. The computer program comprises instructions which, when executed by a computing device, cause the computing device to carry out the method according to the first or second aspect of the invention, or any embodiment thereof. The computer program may be stored on a program readable storage medium.

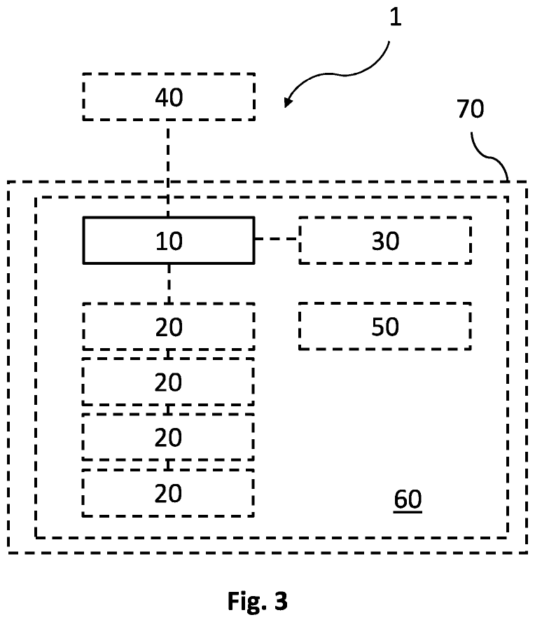

FIG. 3 shows a block diagram of the components of a device according to one embodiment of the invention wherein dashed segments indicate a further embodiment of the invention.

The device 1 is configured for extracting respiratory information from a bio-impedance signal. The device 1 comprises a processor 10 configured to execute the method S0, S0′ according to the first or second aspect of the invention, or any embodiments thereof. The device 1 may comprise other electronics components such as one or more program readable storage mediums 20, a communications module 30, a power source 50 and a circuit board 60. The device 1 may comprise a housing 70 configured to house the processor 10 and other electronics components. The device 1 may also comprise one or more electrodes 40 configured to be arranged on the subject for measuring a bio-impedance, for example in tetra-polar configuration on the subject's torso.

The device 1 may be a wearable patch device.

The one or more program readable storage mediums 20 may be configured to store extracted respiratory information and/or Savtizky-Golay filter parameters, and/or in the case the Savitzky-Golay filter or filters are configured to be adaptively updated, updated Savitkzy-Golay filter parameters.

The communications module 30 may be configured to enable the device 1 to receive and process filter control data, which control data specify control data of the Savitzky-Glay filter or filters.

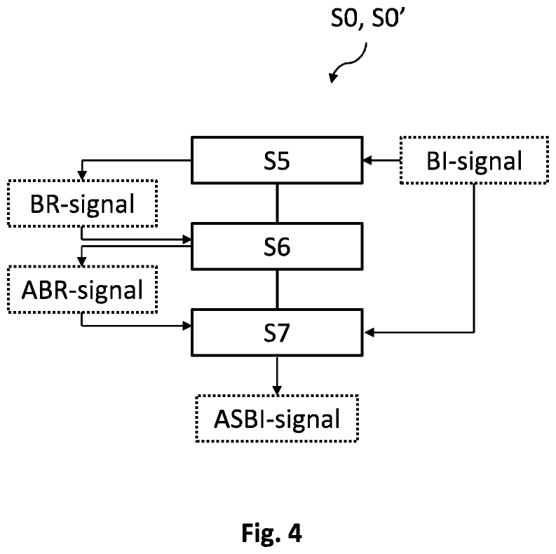

FIG. 4 shows a chart of the method S0, S0′ according to one embodiment of the invention wherein the Savitzky-Golay filter or filters are adaptively updated based on an instantaneous respiratory frequency. In the embodiment shown in FIG. 4, the instantaneous respiratory frequency is estimated from the bio-impedance, BI, signal. The instantaneous respiratory frequency is estimated from the BI-signal as follows. A breath-by-breath breathing rate, BR, is estimated from the bio-impedance, BI, signal via step S5 which results in a BR-signal. A moving average is then performed of the resulting BR-signal with non-overlapping time windows of frame lengths selected within the interval of 15-30 seconds via step S6. The resulting averaged BR-signal is denoted as averaged breathing rate, ABR, signal. A multiplier M is then applied so that at any point in time along the BI-signal, the adaptive frame length of the Savitzky-Golay smoothing filter is equal to M*(1/ABR), thus resulting in an adaptively smoothed bio-impedance, ASBI, signal. Said multiplier M is a value selected in the interval of 0.10-0.30.

In the drawings and specification, there have been disclosed preferred embodiments and examples of the invention and, although specific terms are employed, they are used in a generic and descriptive sense only and not for the purpose of limitation, the scope of the invention being set forth in the following claims.

1. A method for extracting respiratory information from a bio-impedance signal, the method comprising the steps of:
providing a bio-impedance signal of a subject;
filtering the bio-impedance signal using a Savitzky-Golay low-pass filter to provide first respiratory information representing a respiratory effort signal;
wherein
the Savitzky-Golay low-pass filter incorporates a 3rd degree polynomial fit,
wherein the Savtizky-Golay low-pass filter is applied on subsets of data points of the bio-impedance signal which are filtered using a frame length in the interval of 1.1-2 seconds.

2. The method according to claim 1, comprising the step of:
differentiating the respiratory effort signal with a Savitzky-Golay first derivative kernel to provide second respiratory information representing a respiratory flow signal,
wherein the Savitzky-Golay first derivative kernel incorporates a 2nd degree polynomial fit.

3. A method for extracting respiratory information from respiratory measurements of a subject, the method comprising the steps of:
providing a noise filtered respiratory effort signal;
differentiating the noise filtered respiratory effort signal with a Savitzky-Golay first derivative kernel to provide second respiratory information representing a respiratory flow signal,
wherein the Savitzky-Golay first derivative kernel incorporates a 2nd degree polynomial fit,
wherein the Savitzky-Golay first derivative kernel is applied on subsets of datapoints of the respiratory effort signal which are filtered using a frame length in the interval of 0.5-1.5 seconds.

4. The method according to claim 3, wherein the respiratory effort signal is extracted from the respiratory measurement by filtering the bio-impedance signal using a Savitzky-Golay low-pass filter incorporating a 3rd degree polynomial fit.

5. The method according to claim 1, comprising a step of filtering the respiratory effort signal using a high-pass filter to obtain a baseline filtered respiratory effort signal.

6. The method according to claim 5, wherein the high-pass filter has a cutoff frequency in the interval of 0.001-0.1 Hz.

7. The method according to claim 1, wherein the Savitzky-Golay filter or filters are adaptively updated based on an instantaneous respiratory frequency.

8. The method according to claim 7, wherein the instantaneous respiratory frequency is estimated from the bio-impedance signal,
wherein the method incorporates the steps of
detecting a breath-by-breath breathing rate, BR, from the bio-impedance, BI, signal
performing a moving average of the resulting BR-signal with non-overlapping time windows of frame lengths in the interval of 15-30 seconds, which resulting BR-signal is denoted as ABR-signal, and
applying a multiplier M so that at any point in time along the BI-signal, the adaptive frame length of the Savitzky-Golay smoothing filter is equal to M*(1/ABR), thus resulting in an adaptively smoothed bio-impedance, ASBI, signal,
wherein said M is a value selected in the interval of 0.1-0.30.

9. The method according to claim 1, wherein the Savtizky-Golay low-pass filter is applied on subsets of data points of the bio-impedance signal which are filtered using a frame length in the interval of 1.3-1.7 seconds.

10. The method according to claim 1, wherein the Savitzky-Golay first derivative kernel is applied on subsets of datapoints of the respiratory effort signal which are filtered using a frame length in the interval of 0.5-1.5 seconds.

11. A computer program comprising instructions which, when executed by a computing device, cause the computing device to carry out the method according to claim 1.

12. A program readable storage medium storing the computer program according to claim 11.

13. The method according to claim 1, wherein the Savtizky-Golay low-pass filter is applied on subsets of data points of the bio-impedance signal which are filtered using a frame length in the interval of 1.3-2 seconds.

14. The method according to claim 3, wherein the respiratory measurements of a subject are from a bio-impedance signal.

15. The method according to claim 3, wherein the noise filtered respiratory effort signal is a respiratory effort signal extracted from a bio-impedance signal.

16. The method according to claim 5, wherein the high-pass filter has a cutoff frequency in the interval of 0.01-0.05 Hz.

17. The method according to claim 1, wherein the Savitzky-Golay first derivative kernel is applied on subsets of datapoints of the respiratory effort signal which are filtered using a frame length in the interval of 0.8-1.2 seconds.
